(19) United States
(12) Patent Application Publication
(10) Pub. No.: US 2019/0192043 A1
(43) Pub. Date: Jun. 27, 2019

(54) Body Composition Analysis Method and Apparatus

(71) Applicant: TomTom International B.V., Amsterdam (NL)

(72) Inventors: Donald Leckie, Amsterdam (NL); Gary Fullerton, Amsterdam (NL); Davide Zilio, Amsterdam (NL)

(21) Appl. No.: 16/329,382

(22) PCT Filed: Sep. 1, 2017
(86) PCT No.: PCT/EP2017/072008

§ 371 Date: Feb. 28, 2019

(30) Foreign Application Priority Data
Sep. 1, 2016 (GB) 1614882.7

(51) Int. Cl. A61B 5/053; A61B 5/00

(52) U.S. Cl. CPC A61B 5/0537; A61B 5/7275; G16H 50/30; A61B 5/7203; A61B 2562/0209; A61B 5/681

(57) ABSTRACT

The invention relates to a method of providing an indication of a body composition parameter to a user. The method includes receiving a data value indicative of a body composition parameter, providing the data value to a first buffer, the first buffer storing data values received over a first given time period, comparing the data value with a first tolerance range determined from data values stored in the first buffer over the first given time period. If the data value falls within the first tolerance range, the data value is provided to a second buffer, the second buffer storing those data values provided from the first buffer over a second given time period longer than the first given time period. an indication of the body composition parameter is provided to the user based on an average of the data values stored in the second buffer.

### FIELD OF INVENTION

The present invention is concerned with providing a measure or indication of body composition of a user, in particular a measure of body fat and/or muscle.

### BACKGROUND OF THE INVENTION

In recent times there have been great developments in the fields of health and fitness. In general, more people are concerned with living a healthy life and are concerned to keep fit and healthy. With improvements and advancements in available information technology, more information on health and fitness is available to users. Fitness magazines and online sources enable people to be kept up-to-date on the latest medical knowledge and technical advances that can help them maintain a healthy lifestyle. New devices make it easy for people to track their fitness and/or aspects of their life and physical or physiological parameters in their quest to remain or become fit and healthy.

For example, tools are available to enable people to: calculate their body mass index (BMI) and to compare this to healthy values; count amounts of activity, sleep, calories consumed/expended and heart rate and compare these to healthy values; determine blood sugar levels, cholesterol values, etc; and measure parameters, such as impedance, which can be used, for example, to analyse their body composition, e.g. levels of body fat. Devices have developed in line with the available information and the user's desire to identify their own fitness levels.

In addition to access to information and tools via the Internet or the like, there are now many devices on the market that enable a user to track their fitness in a simple and convenient way, such as apps on mobile telephones and wearable fitness trackers, such as wrist-worn devices and watches incorporating tracking, measuring and sensing functions.

Bio-impedance analysis (BIA) is a technique for measuring body composition, e.g. fat, muscle, etc based on user inputs including impedance. BIA determines the electrical impedance provided by the user's body tissue which can then derive a ratio of body fat to body moisture. Simple devices are known for measuring body fat using BIA using electrodes attached to parts of a user's body; such devices have been found to be not sufficiently accurate for absolute one-off measurements but are useful for tracking changes in an individual over time. The accuracy of the readings is, however, affected by a number of factors and can also vary considerably, for any particular user, over the course of a day due to, for example, times when meals are consumed, hydration at any time, and also the location of the measuring electrodes on the user.

Simple devices for measuring body impedance include two electrodes placed on, e.g. the user's two feet; more accurate results have been found using four electrodes on the hands and feet, i.e. two current and voltage electrodes for each hand and foot, or even more electrodes on the user's body.

An impedance measurement circuit comprises a current source, a voltage measurement circuit and a processor. Impedance can be determined using two sensors—a so-called ‘two-point’ system—whereby current from the source is passed through the body whose impedance is to be measured, from one electrode in contact with the body at one location to a second electrode in contact with the body at another location. The voltage measurement circuit measures the voltage drop across the electrodes to determine the impedance.

Accuracy of the impedance measurement can be improved using a ‘four-point’ system which uses an additional pair of electrodes. Current is fed through two ‘feeding’ electrodes and the voltage drop is measured between two ‘measurement’ electrodes. An example of such an impedance measurement can be found in US 2011/0208458 A1.

As mentioned above, there is an increased demand for wearable or easily portable health and fitness monitoring devices. Such four electrode hand/foot devices do not easily lend themselves to a wearable form. In recent times, algorithms and devices have been developed to add BIA analysis and other body parameter measurement and analysis functions to wearable devices such as wrist-worn fitness trackers. One such device and algorithm is taught in US 2016/089053 A1. Impedance is measured using two electrodes or two pairs of electrodes, one in contact with the user's wrist and another on the outer-facing side of the device which the user touches, e.g. with a finger. Touching the outer electrode completes the circuit from the electrode touching the user's wrist to enable a body impedance measurement. A similar device is taught in US 2016/0106337 A1, which uses both two-point and four-point measurements.

As with the devices mentioned above, however, such wearable devices result in inaccuracies due to various factors such as the degree of contact between the electrodes and the user, especially since the electrodes typically present relatively small surfaces. The contact between the relevant electrode(s) and the user's wrist could be affected by hair, sweat, posture, etc. The contact between the relevant electrode(s) and the user's finger could be affected by finger position, skin conductance, etc. Food and water intake of the patient will also affect the measurements and so these will vary even over a given day, for any user, due to factors other than fat levels.

Having a user take measurements at the same time each day can reduce these inaccuracies to some extent; and devices can be configured to alert a user as to when and/or how the measurements should be made, but this reduces the convenience and simplicity to the user and even with such controls, readings are still found to fluctuate.

There is, therefore, a need to provide an algorithm or method of providing a more accurate BIA output without detracting from the convenience and simplicity to the user, and for devices incorporating such algorithms.

### SUMMARY OF THE INVENTION

According to one aspect, the invention provides a method of providing an indication of a body composition parameter to a user, comprising:

obtaining a new measurement indicative of a body composition parameter;

adding the new measurement to a first data set in a first data buffer stored on a data storage device, the first data set including a plurality of previous measurements obtained over a first predetermined time period;

determining a tolerance range based on an average of the measurements in the first data set;

adding the new measurement to a second data set in a second data buffer stored on the data storage device when the new measurement is within the determined tolerance range, the second data set including a plurality of previous measurements obtained over a second predetermined time period longer than the first predetermined time period;

determining an adjusted measurement based on an average of the measurements in the second data set; and

providing data indicative of the adjusted measurement to a device for provision to the user.

The present invention extends to a system for carrying out a method in accordance with any of the aspects or embodiments of the invention herein described. Thus, in accordance with a second aspect of the invention there is provided a system for providing an indication of a body composition parameter to a user, comprising:

means for obtaining a new measurement indicative of a body composition parameter;

means for adding the new measurement to a first data set in a first data buffer stored on a data storage device, the first data set including a plurality of previous measurements obtained over a first predetermined time period;

means for determining a tolerance range based on an average of the measurements in the first data set;

means for adding the new measurement to a second data set in a second data buffer stored on the data storage device when the new measurement is within the determined tolerance range, the second data set including a plurality of previous measurements obtained over a second predetermined time period longer than the first predetermined time period;

means for determining an adjusted measurement based on an average of the measurements in the second data set; and

means for providing data indicative of the adjusted measurement to a device for provision to the user.

As will be appreciated by those skilled in the art, this further aspect of the present invention can and preferably does include any one or more or all of the preferred and optional features of the invention described herein in respect of any of the other aspects of the invention, as appropriate. If not explicitly stated, the system of the present invention herein may comprise means for carrying out any step described in relation to the method of the invention in any of its aspects or embodiments, and vice versa.

The present invention is a computer implemented invention, and any of the steps described in relation to any of the aspects or embodiments of the invention may be carried out under the control of a set of one or more processors. The means for carrying out any of the steps described in relation to the system may be a set of one or more processors.

The methods of the present invention may be carried out by a mobile device. The mobile device has a memory and a set of one or more processors. Such a mobile device can include a device that is designed to be worn by a user, such as a fitness watch, fitness tracker or other wearable sensor, e.g. that can be worn during an exercise activity (running, cycling, swimming, hiking, skiing, weightlifting, etc.), which can track and display information relating to a user's activity levels, such as the heart rate of the user at particular moments during a workout. Accordingly, in some embodiments, the mobile device can include means, such as one or more sensors, for measuring at least one body composition parameter, in addition to means for obtaining and processing the measurements taken by the one or more sensors. In other embodiments, the mobile device can include a mobile phone, tablet device or other computing device, that include means for receiving the measurements of at least one body composition parameter from a remote sensor or sensors, e.g. using a wireless connection. Alternatively the methods of the present invention may be carried out by a server. Other embodiments are envisaged in which the methods of the present invention are performed by a combination of a server and a mobile device. Accordingly, the system of the present invention may comprise a mobile device and/or a server arranged to perform the steps described.

The present invention is directed to providing an indication of a body composition parameter to a user. The body composition parameter will typically be a parameter of the body of the user to which the indication is provided, but it is contemplated that the indication could be provided to a different user as desired. The body composition parameter can be any parameter of a user that is desired to be measured and/or monitored, and can include one or more of: body impedance, e.g. as measured in BIA systems; body fat percentage, e.g. as obtained using a BIA system; and body muscle percentage, e.g. as obtained using a BIA system. Thus, for example, in embodiments of the invention, the new measurement indicative of a body composition parameter

In the invention, a new measurement indicative of a body composition parameter is obtained, e.g. as measured by a sensor or measuring device. The new measurement can be, for example, a data indicative of a body fat percentage and/or a body muscle percentage. The measurement can be derived from a measurement of the impedance of a user's body, or portion of the body, e.g. as measured by a bio-impedance analysis (BIA) device. As discussed above, the sensor or measuring device can be within the computing device that performs the method of the present invention, e.g. with the measured data value be obtained over a wired connection. Alternatively, the sensor or measuring device can be remote from the computing device that performed the method of the present invention, e.g. with the measured data value being obtained over a wireless connection, e.g. WiFi, Bluetooth or similar communication protocol. In preferred embodiments, the new measurement is obtained immediately after the measurement is made by the respective sensor or measuring device, such that data indicative of an adjusted measurement (based on the new measurement) can be provided to a user, e.g. displayed on a display device, immediately after the measurement is made, or at least quickly thereafter. Although it is envisaged that the measurement could be a measurement that occurred at a time in the past.

The new measurement is added to a first data set in a first data buffer stored on a data storage device, e.g. a memory. The first data set includes a plurality of previous measurements obtained over a first predetermined time period. In embodiments, the plurality of previous measurements can include all the measurements taken in the first time period. Alternatively, the first data buffer can include the most recent measurements taken in the first time period, e.g. the most recent 10 or 15 measurements. In other words, the first data buffer can function as a first in-first out (FIFO) buffer such that there is always at most a predetermined number of measurement in the buffer. The first time period can be a certain number of days, such as 2 days; although this number is merely exemplary. The first data buffer can be thought as a short trend buffer.

A tolerance range is determined based on an average of the measurements in the first data set. The average can be any measure of central tendency of the distribution of measurements as desired, such as the mean, e.g. arithmetic mean, harmonic mean, etc, the median, the mode or the like. Although in preferred embodiments, the determined average is the arithmetic mean. The tolerance range defines a range of data values based on, e.g. centred on, the determined average. For example, the tolerance range can be defined by a lower percentile of the distribution, e.g. 48^{th} percentile or similar, and an upper percentile of the distribution, e.g. 52^{nd} percentile or similar. The tolerance range is preferably centred on the determined average, e.g. the 50^{th} percentile, although this does not need to be the case. The tolerance range is used to determine whether an obtained measurement is an outlier, i.e. is statistically different from previous measurements that have recently been received. Since the measurements relate to a parameter of the body, e.g. fat percentage or muscle percentage, it can be assumed the parameter should not change dramatically in the short term, and thus it can be assumed a new measurement that is significantly different from a recent previous measurement is not a ‘good’ or valid measurement and should be ignored. Such outliers can be thought of as ‘bad’ or invalid measurements.

Accordingly, in the present invention, the new measurement is only added to a second data set in a second data buffer stored on the data storage device when the new measurement is within the determined tolerance range. The second data set again includes a plurality of previous measurements, but in contrast to the first data set, these previous measurements have been obtained over a second predetermined time period longer than the first time period. In other words, while the first data buffer can be thought of as a short trend buffer, the second data buffer can be thought of as a long trend buffer. The second time period can again be a certain number of days, such as 10 days; although this number is again merely exemplary. As will be appreciated, since new measurements are only added to the second data buffer when they are deemed valid, i.e. are within the tolerance range determined at the time of each measurement (based on the measurements in the first data buffer at the time of the measurement), and thus the second data buffer preferably only includes valid measurements. In embodiments, the plurality of previous measurements can include all the valid measurements taken in the second time period. Alternatively, the second data buffer can include the most recent valid measurement taken in the second time period. In other words, the second data buffer can function as a first in-first out (FIFO) buffer such that there is always at most a predetermined number of valid measurements in the buffer.

In the present invention, an adjusted measurement is determined based on an average of the measurements in the second data set. The average can be any measure of central tendency of the distribution of measurements as desired, such as the mean, e.g. arithmetic mean, harmonic mean, etc, the median, the mode or the like. Although in preferred embodiments, the determined average is the arithmetic mean. The adjusted measurement can be the determined average, although in embodiments, and as discussed below, the adjusted measurement may be based on, but not equal, the determined average. For example, it has been recognised that the body fat percentage and the body muscle percentage do not typically vary by more than a predetermined amount within a given period of time. More specifically, it has been found that the body fat percentage and body muscle percentage, in most cases, do not vary by more than 1% in a 24 hour period. This knowledge can be used, in embodiments, to determine the adjusted measurement. For example, when the average of the measurements in the second data set is different from previous adjusted measurements returned in a given period of time by more than a predetermined amount, then the average value is clipped or capped to a value equal to the previous measurement plus or minus (as required) the predetermined amount. For example, in an embodiment of the invention, a determination is made as to whether the average is more than 0.5% above or below any returned adjusted measurements made in the last 12 hours, and, if this is determined to be the case, the average is increased or decreased as required.

Data indicative of the adjusted measurement is provided to a device for provision to the user, e.g. in response to the measurement that has just been made, e.g. by the user interacting with one or more electrodes of BIA sensor. In embodiments, the data indicative of the adjusted measurement is the value of the adjusted measurement. For example, adjusted measurement can be transmitted to another device, e.g. using a communications device, such as a wireless communications device (e.g. Bluetooth, WiFi, etc) for display, analysis, etc, e.g. to a web site or a user's mobile phone or other device. Additionally, or alternatively, the adjusted measurement can be displayed to the user using a display device of the device on which the method was performed, e.g. a wearable device such as a wrist-worn fitness tracker or sports watch.

In embodiments of the invention, the first and/or second data buffers can be cleared if a new measurement is not obtained in a predetermined period of time, such as 14 days. As will be appreciated, the period of time that triggers a reset of the data buffer can differ between the first and second data buffers as desired. The data buffers, and thus associated statistics based on the first and second data sets, are reset following a certain period of inactivity, such that subsequently received new measurements are not adversely influenced by out-of-date measurements.

It will be appreciated that the methods in accordance with the present invention may be implemented at least partially using software. It will thus be seen that, when viewed from further aspects and in further embodiments, the present invention extends to a computer program product comprising computer readable instructions adapted to carry out any or all of the method described herein when executed on suitable data processing means. The invention also extends to a computer software carrier comprising such software. Such a software carrier could be a physical (or non-transitory) storage medium or could be a signal such as an electronic signal over wires, an optical signal or a radio signal such as to a satellite or the like. Accordingly, in accordance with another aspect of the invention, there is provided a computer program product, e.g. computer software, comprising instructions which, when executed by one or more processors of a system, cause the system to perform the method of any of the aspects and embodiments discussed above. The computer program product can be stored on a non-transitory computer readable medium.

Regardless of its implementation, a mobile or wearable device used in accordance with the present invention may comprise a processor, memory, and optionally one or more sensors for measuring body composition. The processor and memory cooperate to provide an execution environment in which a software operating system may be established. One or more additional software programs may be provided to enable the functionality of the device to be controlled, and to provide various other functions. The device may comprise one or more output interfaces by means of which information may be relayed to the user. The output interface(s) may include one or more of a visual display device and speaker for audible output. The device may comprise input interfaces including one or more physical buttons to control on/off operation or other features of the apparatus.

The present invention in accordance with any of its further aspects or embodiments may include any of the features described in reference to other aspects or embodiments of the invention to the extent it is not mutually inconsistent therewith.

### BRIEF DESCRIPTION OF THE FIGURES

Various embodiments will now be described, by way of example only, and with reference to the accompanying drawings in which:

FIG. 1A is a perspective view of a wrist-worn activity tracker that can incorporate the invention;

FIG. 1B is an alternative perspective view of the activity tracker of FIG. 1A shown the inside or skin-facing side of the device;

FIG. 2 is a flow diagram showing a method according to an embodiment of the invention;

FIG. 3 is a schematic diagram of the various features and components that can be provided in an activity or fitness tracker; and

FIG. 4 is a chart showing the data values and the smoothed output using the method of the invention.

### DETAILED DESCRIPTION OF THE FIGURES

The embodiments below relate to the invention incorporated in a wrist-worn or other wearable device such as a sports watch, or activity or fitness tracker. The invention can, however, be incorporated in other devices such as another mobile device, such as a mobile phone, or on a web server receiving the data values from another device such as those listed here.

Referring to FIGS. 1A and 1B, the invention may be incorporated in a wrist-worn tracker comprising a wrist strap 1 and a tracker module 2 attached to, fitted into, mounted on or in or detachably mounted to the strap 1. The strap may be an elastic or stretchable strap or may be an adjustable strap with a fastener/buckle 3.

In embodiments, the tracker module 2 incorporates a processor 202 (as shown in FIG. 3). The processor may be programmed to perform the smoothing method of the invention. In the preferred embodiment the tracker module incorporates sensor means, described further below, which obtain body signals or measurements from which a body parameter can be calculated and these are then smoothed by the method of the invention, in this embodiment in the same processor. In the embodiment shown and described, the actual indication of the body composition parameter generated by the smoothing method is transmitted to another device for display, analysis, etc rather than being displayed on the display 4 of the activity tracker; the activity tracker can, however, provide an indication that the smoothing process has been completed e.g. by means of a tick icon (described further below) or that the process has failed (e.g. by a cross icon on the display). In other embodiments, however, the actual smoothed indication of the body composition parameter, e.g. percentage body fat of the user, can be displayed on the display 4.

As mentioned above, in the embodiment shown, the activity tracker includes sensor means for obtaining the body measurements/signals for calculating the data values. In other embodiments, though, the user could have a separate device or sensor/monitor to obtain the data values which could then be transmitted to the activity tracker to perform the smoothing process and transmit and/or display the results.

In this embodiment, the sensor means is provided on the device and is in the form of a pair of voltage/current sensors or electrodes 50, 51. One electrode 50 is on the inside of the device so that it comes into contact with the wearer's wrist in use. The other electrode 51 is on the outer-facing side of the tracker. To complete a loop between the two electrodes and through the wearer's body for measuring body parameters, the user places a finger on the outer electrode 50. A measuring current then flows from one electrode to the other through the wearer's body to measure a body parameter such as, in the embodiment described, impedance. Electrodes 50 and 51 are usually, in fact, electrode pairs each comprising an input electrode and an output electrode. A measure of body impedance is obtained as is known in the art; see, for example, US 2016/0089053 A1.

As described above, impedance can be measured using a two-point or a four-point system. If four electrodes are used, these may be provide as two pairs of side-by-side electrodes or, as shown, as two pairs of concentric electrodes. In one example, even where four electrodes are provided, one electrode on each side of the device (FIGS. 50, 51, 52 and 53) is used to determine impedance. The other electrodes 51 and 53 may be part of a feedback system, e.g. to take account of component losses in the system and provide a more accurate reading. In other embodiments, all four electrodes 50, 51, 52 and 53 are used in a four-point measuring system.

Based on the impedance measurement and using other user-specific inputs such as weight, age, height, gender a body composition parameter is calculated preferably using known BIA algorithms. The body composition parameter may be percentage fat, percentage muscle, the amount of fluid/water in the body, muscle strength.

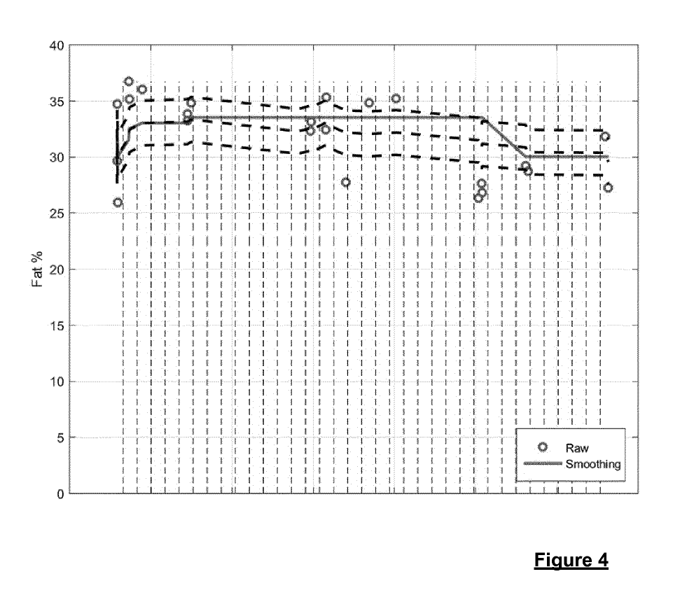

As mentioned above, such measurements may be inaccurate and inconsistent for various reasons. The smoothing method of the invention processes the data values obtained by e.g. the BIA algorithm and provides a smoothed indication of the body composition parameter. This can be seen in the chart of FIG. 4. In this chart, each vertical dotted line represents a new day. The circles represent the data values, the dashed lines indicate the average and the range boundaries for the short trend buffer and the continuous line represents the smoothed output of the long trend buffer.

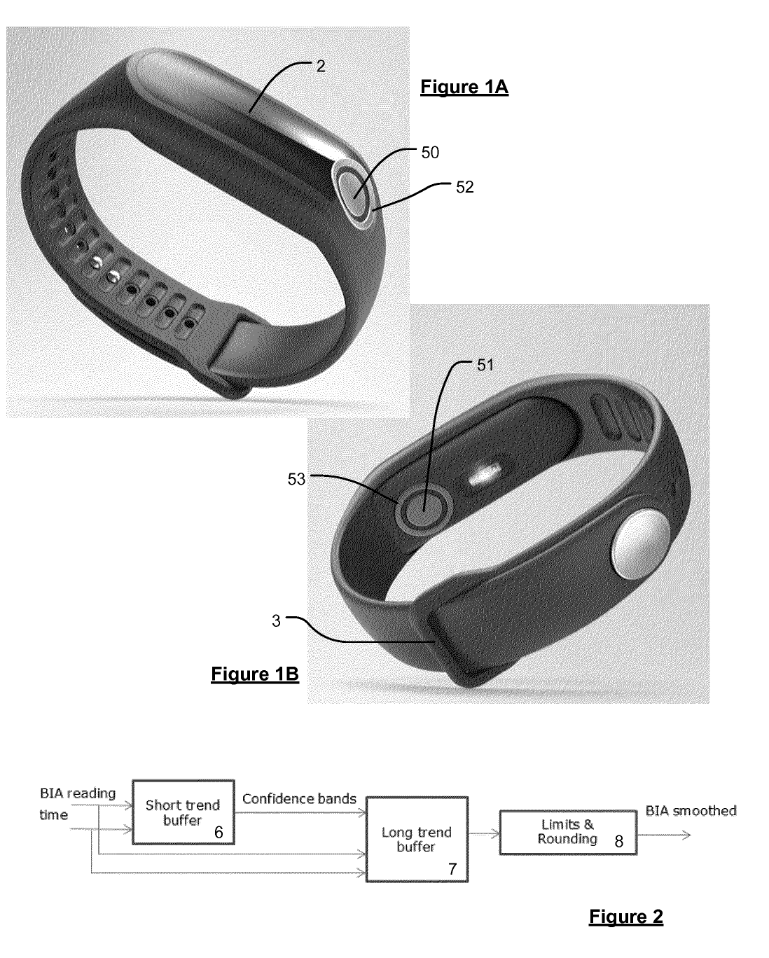

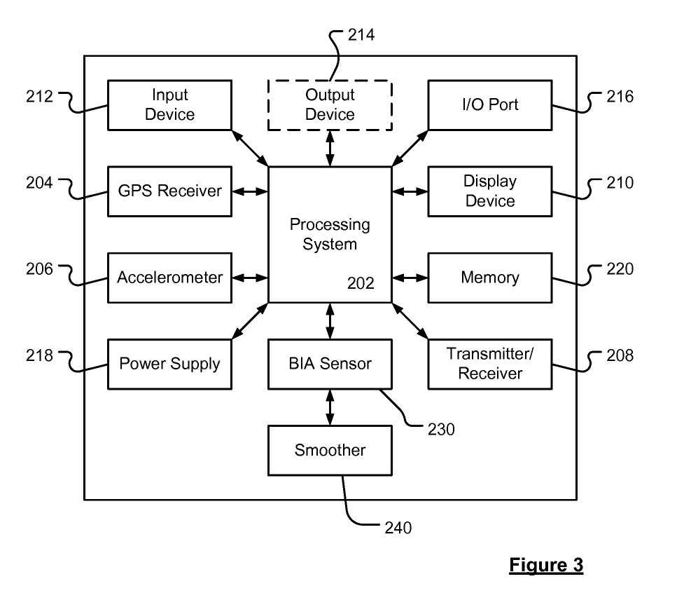

FIG. 2 is a flow diagram showing the method of the invention which, in the embodiment described, is performed in the processor of the tracker module 2.

The data inputs include a data value input which may be a result from the BIA process e.g. fat percentage or muscle and, as a second input, time. These are provided to a short trend buffer 6 which removes any outlier values i.e. those data values that exceed a tolerance range e.g. a range centred about an average value of the data values stored in the short trend buffer over a period of time e.g. a few days or a relatively small number of measurements e.g. 10. Those values falling within the tolerance band—i.e. the ‘good’ values—are provided to the long trend buffer 7.

The long trend buffer stores the ‘good’ values obtained over a period of time e.g. several days and provides an average of these values as an output as an indication of the body composition parameter, for transmission to another device/location or, for display on the device display 4.

The output indication may, before being transmitted/displayed, be subject to a limiting/rounding process 8, whereby fluctuations over a given period of time, e.g. 12 or 24 hours, or a given number of measurements, are only output if they do not fluctuate more than a given percentage e.g. 0.5%, 1%, etc. or, if they do so vary, they are cropped to the maximum variation e.g. 1%.

Preferably, the buffers are reset at regular intervals e.g. every 14 days

FIG. 3 shows an example of the processing capabilities of a fitness tracker. The tracker module 2 includes a processor 202 which communicates with various function modules including input device 212, output device 214, I/O port 216,

A display module 210, memory 220, GPS module 204, power supply 218, transmitter/ receiver 206, BIA module 230 and smoothing module 240. Of course, activity trackers or other wearable devices may have more or fewer functions.

Whilst the smoothing method of the first aspect of the invention, described above, can be used in a wide range of devices, it has found particular application in such wrist-worn devices which preferably incorporate the ‘finger’ sensor or electrode/electrode pair mentioned above.

It will be appreciated that whilst various aspects and embodiments of the present invention have been described, the scope of the present invention is not limited to the described embodiments but, rather, is defined by the claims.

1. A method of providing an indication of a body composition parameter to a user, the method comprising:
receiving a data value indicative of a body composition parameter;
providing the data value to a first buffer, the first buffer storing data values received over a first given time period;
comparing the data value with a first tolerance range determined from data values stored in the first buffer over the first given time period;
if the data value falls within the first tolerance range, providing the data value to a second buffer, the second buffer storing those data values provided from the first buffer over a second given time period longer than the first given time period; providing an indication of the body composition parameter to the user based on an average of the data values stored in the second buffer.

2. The method of claim 1, wherein the body composition parameter comprises one of body impedance, percentage body fat or percentage muscle.

3. The method of claim 1, further comprising means for comparing values of the indication of the body composition parameter with a fluctuation threshold and excluding values that exceed the fluctuation threshold from being provided to the user.

4. The method of claim 1, wherein the indications are transmitted to a remote device for providing to the user.

5. The method of claim 1, wherein the indication is provided to the user via a display worn by the user.

6. The method of claim 1, wherein the data values are received from a device worn by the user.

7. The method of claim 1, further comprising obtaining a measure of impedance of the body of the user and deriving the body composition parameter from the measure of impedance.

8. The method of claim 7, wherein the body composition parameter is derived using bio-impedance analysis.

9. A device comprising a processor configured to perform a method of providing an indication of a body composition parameter to a user, the method comprising:
receiving a data value indicative of a body composition parameter;
providing the data value to a first buffer, the first buffer storing data values received over a first given time period;
comparing the data value with a first tolerance range determined from data values stored in the first buffer over the first given time period;
if the data value falls within the first tolerance range, providing the data value to a second buffer, the second buffer storing those data values provided from the first buffer over a second given time period longer than the first given time period; providing an indication of the body composition parameter to the user based on an average of the data values stored in the second buffer.

10. The device of claim 9 comprising a wearable device comprising the processor.

11. The device of claim 9, further comprising first, and second electrodes for making contact with the body of the user and by means of which the body composition parameter can be calculated.

12. The device of claim 11 further comprising a current source to provide current to one of the first and second electrodes and a voltage measuring means for measuring a voltage drop across the first and second electrodes from which impedance is calculated to provide the data value.

13. The device of claim 11, wherein the first electrode comprises a first pair of electrodes and the second electrode comprises a second pair of electrodes.

14. The device of claim 13, wherein the electrodes of the first pair are arranged either side-by-side or concentrically and the electrodes of the second pair are arranged either side-by-side or concentrically.

15. The device of claim 11, wherein one of the electrodes is further configured to operate in a mode to control functions of the device other than calculation of the body composition parameter.

16. A wearable device comprising: a body parameter measurement circuit comprising a first electrode on a body side of the device arranged to electrically contact the wearer's body when the device is worn, a second electrode on an opposite side of the device arranged to be touched by the wearer so as to complete an electric circuit from the first electrode, through the wearer's body, to the second electrode; the device further comprising a display and a processor configured to perform a plurality of wearer-activity-related functions; wherein the second electrode is arranged to operate in a first mode as an electrode of the measurement circuit and in a second mode as a touch input means to provide an input to the processor to control another function of the device.

17. The device of claim 16, comprising a pair of first electrodes arranged side-by-side or concentrically and a pair of second electrodes arranged side-by-side or concentrically.

18. The device of claim 16, wherein the measurement circuit is an impedance measurement analysis circuit arranged to determine a body composition parameter from the measured impedance.

19. The device of claim 16, wherein the measurement circuit comprises a bio-impedance analysis circuit arranged to determine a body composition parameter from the measured impedance.

20. The device of claim 16, comprising a processor 9 configured to perform a method of providing an indication of a body composition parameter to a user, the method comprising:
receiving a data value indicative of a body composition parameter;
providing the data value to a first buffer, the first buffer storing data values received over a first given time period;
comparing the data value with a first tolerance range determined from data values stored in the first buffer over the first given time period;
if the data value falls within the first tolerance range, providing the data value to a second buffer, the second buffer storing those data values provided from the first buffer over a second given time period longer than the first given time period; providing an indication of the body composition parameter to the user based on an average of the data values stored in the second buffer, wherein the measurement circuit provides the data value.
